(19) United States
(12) Patent Application Publication
(10) Pub. No.: US 2026/0053892 A1
(43) Pub. Date: Feb. 26, 2026

(54) Induction and Expansion of Regulatory T Cells

(71) Applicant: Board of Regents of the University of Nebraska, Lincoln, NE (US)

(72) Inventors: Howard E. Gendelman, Omaha, NE (US); Milica Markovic, South San Francisco, CA (US); R. Lee Mosley, Omaha, NE (US)

(21) Appl. No.: 19/106,097

(22) PCT Filed: Jun. 23, 2023
(86) PCT No.: PCT/US2023/026042

§ 371 Date: Feb. 24, 2025

Related U.S. Application Data
(60) Provisional application No. 63/400,793, filed on Aug. 25, 2022.

(51) Int. Cl. A61K 38/19; A61K 38/20; A61P 25/16; C12N 5/077; C12N 5/0783

(52) U.S. Cl. CPC A61K 38/193; A61K 38/2013; A61P 25/16; C12N 5/0637; C12N 5/0652; C12N 2501/22; C12N 2501/2302

(57) ABSTRACT

The present invention provides novel compositions and methods for inducing and/or activating regulatory T cells (Treg). Wherein a method of treating, inhibiting, and/or preventing a disease or disorder in a subject in need thereof, said method comprising administering at least two immune modulators to said subject, is disclosed.

This application claims priority under 35 U.S.C. § 119 (e) to U.S. Provisional Patent Application No. 63/400,793, filed Aug. 25, 2022. The foregoing application is incorporated by reference herein.

This invention was made with government support under Grant No. R01-NS034239 awarded by the National Institutes of Health. The government has certain rights in the invention.

### FIELD OF THE INVENTION

The present invention relates generally to regulatory T cells. More specifically, the present invention provides compositions and methods for inducing regulatory T cells and treating neurodegenerative diseases such as Parkinson's disease.

### BACKGROUND OF THE INVENTION

CD4+ regulatory T cells (Tregs) play critical roles in the maintenance of immunological homeostasis and the limitation of pathogenic defense mechanisms to restrict responses that might lead to immune-mediated pathologies. Tregs are responsible for maintaining active immune tolerance and exert their immunomodulatory activities through several mechanisms that include suppressive and anti-inflammatory cytokines, IL-10 and transforming growth factor-β. Tregs also induce cytolysis of hyper-reactivate inflammatory cells, suppress antigen presentation by dendritic cells, induce T-cell anergy in naive T cells, alter effector T (Teff) cell metabolic responses, and serve as a sink for T cell growth factors (Reynolds, et al. (2010) J. Immunol., 184:2261-2271; Chinen, et al. (2016) Nat. Immunol., 17:1322-1333; Kalekar, et al. (2007) J. Immunol., 198:2527-2533; Ren, et al. (2007) Cell Death Differ., 14:2076-2084). Tregs also serve to control Teff-associated neurotoxicity. Based on Treg functions, therapies have emerged which increase the number or function of Tregs, such as for inflammatory-mediated autoimmune diseases (Schutt, et al. (2018) Mol. Neurodegener., 13:26). Deficits in Tregs and Treg functions have been demonstrated in several autoimmune disorders such as multiple sclerosis, type-1 diabetes, and inflammatory bowel disease (Dominguez-Villar, et al. (2018) Nat. Immunol., 19:665-673). Parkinson's Disease (PD) patients also exhibit reduced Treg numbers and/or function as well as increased numbers of Teffs (Saunders, et al. (2012) J. Neuroimmune Pharmacol., 7:927-938; Kustrimovic, et al. (2018) J. Neuroinflammation 15:205; Thome, et al. (2021) NPJ Parkinsons Dis. 7:41).

Adoptive transfer of Tregs to MPTP-intoxicated animals, a model for PD, is sufficient to restore immunological homeostasis, attenuate neuroinflammation, and provide protection for dopaminergic neurons in the substantia nigra and striatum (Reynolds, et al. (2010) J. Immunol., 184:2261-2271). However, the dysfunctions of the endogenous Treg source pool may be difficult to overcome and may affect post-administration survival and activity, thus rendering diminished capacities for sustained clinical benefit leading to continued disease progression after transfer (He, et al. (2013) Wiley Interdiscip. Rev. Syst. Biol. Med., 5:153-180). Immunoregulatory peptides and growth factors, such as vasoactive intestinal peptide (VIP) and granulocyte-macrophage colony-stimulating factor (GM-CSF) are immunomodulatory agents that induce or augment Treg numbers (Schwab, et al. (2020) Neurobiol. Dis., 137:104760; Olson, et al. (2021) Biomaterials 272:120786; Kosloski, et al. (2013) J. Neuroimmunol., 265:1-10; Olson, et al. (2015) J. Neurosci., 35:16463). Administration of either agent before or at the time of neuronal insult in MPTP models increases Treg function, attenuates microglial activation and can protect dopaminergic neurons eliminated due to neurodegeneration (Olson, et al. (2021) Biomaterials 272:120786; Mosley, et al. (2019) Front. Cell Neurosci., 13:421). In recent clinical trials, administration of human recombinant GM-CSF (sargramostim, Leukine®; 3 or 6 μg/kg/day) was found to be safe and well-tolerated at low doses, increased Treg numbers, and modestly improved Unified Parkinson's Disease Rating Scale (UPDRS) scores (Gendelman, et al. (2017) NPJ Parkinsons Dis., 3:10; Olson, et al. (2021) EBioMedicine 67:103380). Notably, VIP and GM-CSF exhibit relatively short half-lives (e.g., six-hour half-life of GM-CSF) that present limitations for their clinical use in terms of the overall dose, dose frequency, and bioavailability. Accordingly, compositions and methods for inducing more robust Treg responses are needed.

### SUMMARY OF THE INVENTION

In accordance with the instant invention, compositions and methods for treating, inhibiting, and/or preventing a disease or disorder are provided, particularly a disease or disorder associated with dysregulation of Tregs and/or low levels of Treg numbers and/or function. The instant invention also encompasses compositions and methods for increasing and/or inducing Tregs in cells and/or a subject. The methods can be performed in vitro, in vivo, and/or ex vivo. In certain embodiments, the method comprises administering at least two immune modulators to the subject or cells (e.g., immune cells). In certain embodiments, the disease or disorder is selected from the group consisting of a neurodegenerative disease, an autoimmune disease, an inflammatory disease, and cancer. In certain embodiments, the disease or disorder is Parkinson's disease. In certain embodiments, the immune modulators are selected from the group consisting of GM-CSF, GM-CSF analogs, TGF-β, interleukin (IL)-10, IL-2, glatiramer acetate, anti-CD3, bee venom phospholipase A₂ (bvPLA₂), anti-CD28, rapamycin, histone deacetylase inhibitors, vasoactive intestinal peptide (VIP), VIP analogs, and VIP receptor-2 agonists. The immune modulators may be administered simultaneously and/or sequentially. In certain embodiments, one of the immune modulators is GM-CSF. In certain embodiments, one of the immune modulators is IL-2, particularly low dose IL-2. In certain embodiments, the at least two immune modulators comprise GM-CSF and IL-2. In certain embodiments, the method comprises administering GM-CSF to the subject or cells and then administering IL-2 to the subject or cells.

In accordance with another aspect of the instant invention, compositions comprising at least two immune modulators and at least one pharmaceutically acceptable carrier are provided. In certain embodiments, the immune modulators are selected from the group consisting of GM-CSF, GM-CSF analogs, TGF-β, IL-10, IL-2, glatiramer acetate, anti-CD3, bee venom phospholipase A₂ (bvPLA₂), anti-CD28, rapamycin, histone deacetylase inhibitors, vasoactive intestinal peptide (VIP), VIP analogs and VIP receptor-2 agonists. In certain embodiments, the composition comprises GM-CSF and IL-2.

### BRIEF DESCRIPTIONS OF THE DRAWING

FIG. 1 provides a graph of CD4+CD25+ Tregs in blood samples from mice treated with PBS (control), GM-CSF, IL-2, or GM-CSF and IL-2.

FIG. 2 provides a graph of CD4+CD25+ Tregs in lymph nodes from mice treated with PBS (control), GM-CSF, IL-2, or GM-CSF and IL-2.

FIG. 3 provides a graph of CD4+CD25+FOXP3+ Tregs in spleen from mice treated with PBS (control), GM-CSF, IL-2, or GM-CSF and IL-2.

FIG. 4 provides a graph of CD4+CD25+FOXP3+ Tregs in blood samples from mice treated with PBS (control), GM-CSF, IL-2, or GM-CSF and IL-2.

FIG. 5 provides a graph of CD25+CD127− Tregs in blood samples from mice treated with PBS (control), GM-CSF, IL-2, or GM-CSF and IL-2.

FIG. 6 provides a graph of CD25+CD127− Tregs in spleen from mice treated with PBS (control), GM-CSF, IL-2, or GM-CSF and IL-2.

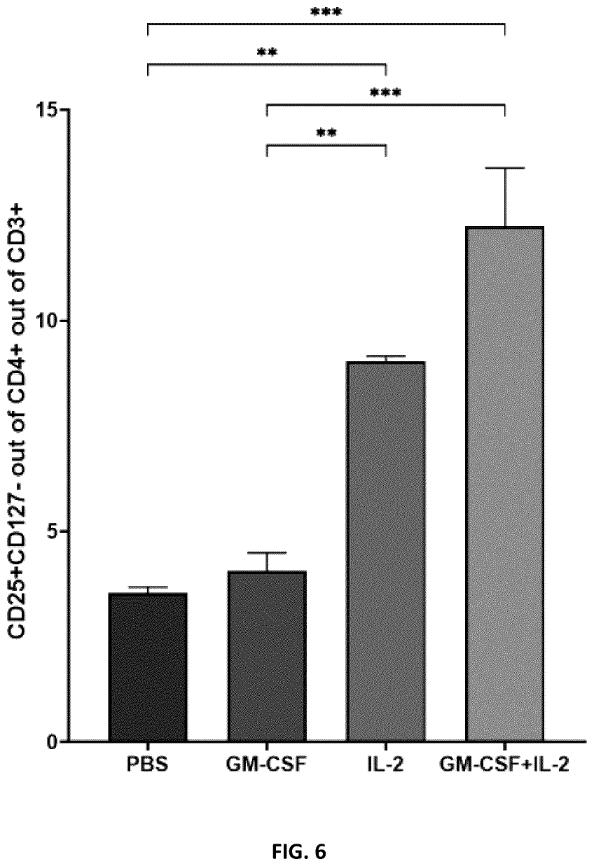

FIG. 7 provides a graph of CD25+ICOS+ (inducible costimulator) Tregs in blood samples from mice treated with PBS (control), GM-CSF, IL-2, or GM-CSF and IL-2.

FIG. 8 provides a graph of CD25+ICOS+ Tregs in spleen from mice treated with PBS (control), GM-CSF, IL-2, or GM-CSF and IL-2.

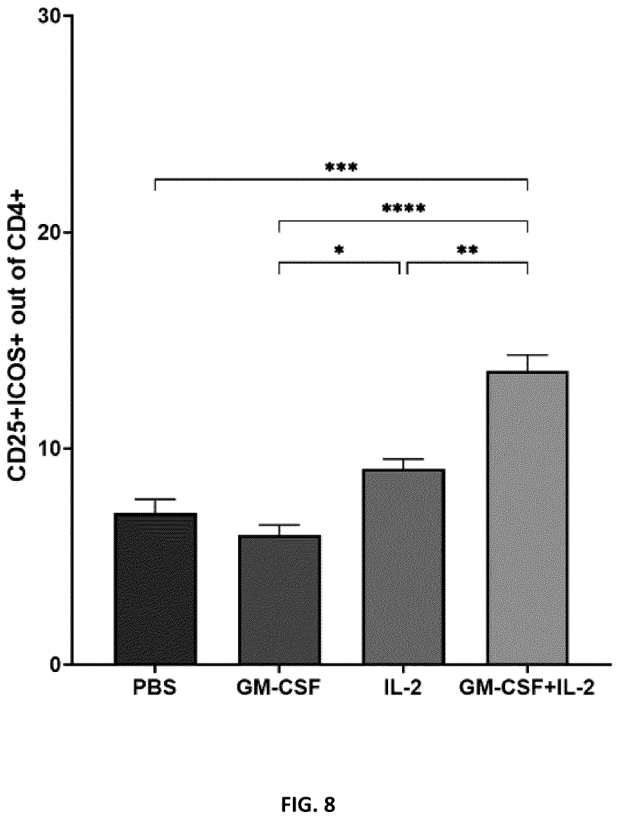

FIG. 9 provides a graph of CD25+GITR+ (glucocorticoid-induced tumor necrosis factor-related receptor) Tregs in lymph nodes from mice treated with PBS (control), GM-CSF, IL-2, or GM-CSF and IL-2.

FIG. 10 provides a graph of CD25+CD39+ Tregs in spleen from mice treated with PBS (control), GM-CSF, IL-2, or GM-CSF and IL-2.

FIG. 11 provides a graph of CD25+CD62L− Tregs in spleen from mice treated with PBS (control), GM-CSF, IL-2, or GM-CSF and IL-2.

FIG. 12 provides graphs of the in vitro induction of isolated Tregs from mice treated with PBS (control), GM-CSF, IL-2, or GM-CSF and IL-2. The isolated Tregs were cultured with IL-2 at 0.1 U/ml, 1 U/ml, or 10 U/ml.

FIG. 13 provides a graph of the percentage of FoxP3+ cells of the in vitro IL-2-induced CD4+CD25+ cells.

### DETAILED DESCRIPTION OF THE INVENTION

Current treatment options of neurodegenerative and autoimmune diseases at best treat the disease symptoms, but are unsuccessful in slowing the progression or addressing causative aspects of these diseases. In disorders whereby immune tolerance is evaded, the subject's immune system recognizes self as foreign and attacks. Herein, compositions and methods are provided which induce, re-establish, and/or maintain immune tolerance such that the subject's immune system recognizes self as not foreign and does not attack. More specifically, the instant invention provides compositions and methods which induce and/or increase Tregs.

Immune tolerance is activated by Tregs. Tregs signal the immune system to dampen pro-inflammatory responses and become tolerant to self- or modified self-antigens that stimulate pro-inflammatory responses. In the case of neurodegenerative disorders, Tregs mitigate the neuroinflammation that drives neuronal loss in diseases such as Parkinson's disease and Alzheimer's disease. Therefore, induction of Treg levels or activity would lead to control of disease-initiating inflammatory activities and mitigate disease progression. The regulatory mechanisms induced in peripheral lymphoid organs by Tregs improve disease outcomes. To such ends, this invention demonstrates synergistic effects with two immune modulators to boost Treg numbers and the Treg phenotype leading to a sustained suppressive immune response for neurodegenerative diseases (e.g., Parkinson's disease, Alzheimer's disease) and autoimmune disorders.

The present invention describes compositions and methods of treating neurodegenerative diseases and autoimmune diseases through the transformation and/or induction of regulatory T cells. Specifically, Tregs are induced and/or activated by treatment with two or more immune modulators. These immune modulators exert synergistic effects in the induction and/or activation of Tregs, particularly in patients suffering from diseases in which immune tolerance is evaded. The present invention provides for the preferential expansion of Treg populations and activity as a treatment for chronic inflammatory disorders without causing overall immune activation. The synergy with the two or more immune modulators leads to reduced doses and lowered toxicities. In certain embodiments, two or more immune modulators are administered to a patient for the treatment of a disease. The immune modulators may be administered at the same time or they may be administered at different times. In certain embodiments, the compositions and the methods of the instant invention comprise GM-CSF and IL-2.

In accordance with the instant invention, compositions and methods are provided for treating, inhibiting, and/or preventing a disease or disorder in a subject. In certain embodiments, the disease or disorder can be treated by increased Tregs and/or increased Treg function, activation, and/or induction. In certain embodiments, the disease or disorder is a neurodegenerative disease (e.g., Parkinson's disease), autoimmune disorder (e.g., inflammatory-mediated autoimmune disease), inflammatory disease, or cancer. In certain embodiments, the disease or disorder comprises neuroinflammation. In certain embodiments, the disease or disorder is a neurodegenerative disease. Examples of neurodegenerative disease include, without limitation, Alzheimer's disease, Parkinson's disease, Parkinson's related disorders, Lewy Body disease, amyotrophic lateral sclerosis, prion disease, and Huntington's disease. In certain embodiments, the neurodegenerative disease is Parkinson's disease. Examples of autoimmune disorders include, without limitation: multiple sclerosis, type-1 diabetes, and inflammatory bowel disease.

In certain embodiments, the method comprises administering at least two immune modulators to the subject. Examples of immune modulators include, without limitation: GM-CSF (e.g., human recombinant GM-CSF, molgramostim, sargramostim), GM-CSF analogs, TGF-β, IL-10, IL-2, glatiramer acetate, anti-CD3 antibodies, anti-CD28 antibodies, bee venom phospholipase A₂ (bvPLA₂), rapamycin, histone deacetylase inhibitors, vasoactive intestinal peptide (VIP), VIP analogs and VIP receptor-2 (VIPR2, also known as VPAC2) agonists (e.g., LBT-3627). The immune modulators may be of the species of the subject being treated (e.g., human). In certain embodiments, at least one of the immune modulators is GM-CSF. In certain embodiments, at least one of the immune modulators is IL-2. In certain embodiments, the at least two immune modulators comprise GM-CSF and IL-2. In certain embodiments, the IL-2 is a low dose IL-2. In certain embodiments, a low dose IL-2 is less than 5 million international units per day (MIU/day), less than 4 MIU/day, less than 3 MIU/day, less than 2 MIU/day, or less than 1 MIU/day. In certain embodiments, a low dose IL-2 is about 0.1 to about 5 MIU/day, about 0.5 to about 5 MIU/day, about 0.5 to about 3 MIU/day, or about 1 to about 3 MIU/day.

Immune modulators may comprise a protein, peptide, gene, small molecule, or oligonucleotide (e.g., mRNA or DNA). In certain embodiments, the immune modulator is a protein or polypeptide. In certain embodiments, the immune modulator is a nucleic acid molecule encoding the protein immune modulator. In certain embodiments, the nucleic acid molecule(s) encoding the protein immune modulator(s) is contained within a vector(s) (e.g., expression vector(s)) which may be administered to the subject. Examples of vectors for expressing the molecules of the invention include, without limitation, plasmids and viral vectors (e.g., adeno-associated viruses (AAVs), retroviruses, and lentiviruses). The expression vectors of the instant invention may employ a strong promoter, a constitutive promoter, and/or a regulated promoter. In certain embodiments, the promoter is cell-type specific. In certain embodiments, the nucleic acid molecules are expressed transiently.

In certain embodiments, the immune modulator(s) is formulated or packaged in a delivery vehicle. Examples of delivery vehicles include, without limitation: liposomes, dendrimers, microparticles, and/or nanoparticles. In certain embodiments, the formulation or delivery vehicle in a slow-release formulation or delivery vehicle.

In certain embodiments, at least one of the immune modulators is GM-CSF. In certain embodiments, at least one of the immune modulators is IL-2. In certain embodiments, at least GM-CSF and IL-2 are administered. In certain embodiments, the immune modulators are administered to the subject in a composition(s) further comprising at least one carrier, particularly at least one pharmaceutically acceptable carrier. In certain embodiments, the method further comprises administering at least one other neurodegenerative disease therapeutic.

The immune modulators may be administered at the same time or they may be administered at different times (e.g., sequentially). In certain embodiments, the at least two immune modulators are administered at the same time (e.g., in the same composition). In certain embodiments, the at least two immune modulators are administered sequentially. In certain embodiments, the at least two immune modulators are administered at least partially at the same time (e.g., their administration time frames overlap at least partially). In certain embodiments, the immune modulators are administered in the same composition when administered at the same time. In certain embodiments, the immune modulators are administered in separate compositions (e.g., one immune modulator per composition) when administered at the same time.

In certain embodiments, the method comprises administering a first immune modulator to the subject for a first period of time (e.g., from the first to last administration of the first immune modulator) and a second immune modulator to the subject for a second period of time (e.g., from the first to last administration of the second immune modulator). Generally, the first and second immune modulators are not the same immune modulator. In certain embodiments, the method further comprises administering a third or more immune modulator at a third or more period of time. In certain embodiments, the first period of time at least partially or completely overlaps with the second period of time. In certain embodiments, the first period of time does not overlap with the second period of time. In certain embodiments, the first period of time is before or at least begins before the second period of time. In certain embodiments, the first period of time is before or prior to the second period of time and does not overlap with the second period of time. In certain embodiments, the second immune modulator is IL-2 and is administered, at least partially or completely, after or later than the first immune modulator. In certain embodiments, the first immune modulator is GM-CSF and is administered, at least partially or completely, prior to or before the second immune modulator. In certain embodiments, the first immune modulator is GM-CSF, the second immune modulator is IL-2, and GM-CSF is administered, at least partially or completely, prior to or before the administration of IL-2.

When the first period of time does not overlap with the second period of time, the time in between the first period of time and the second period of time may be less than 2 weeks, less than one week, less than 6 days, less than 5 days, less than 4 days, less than 3 days, less than 2 days, or less than one day. In certain embodiments, the first period of time is less than 10 weeks, less than 9 weeks, less than 8 weeks, less than 7 weeks, less than 6 weeks, less than 5 weeks, less than 4 weeks, less than 3 weeks, less than 2 weeks, or less than 1 week. In certain embodiments, the second period of time is less than 10 weeks, less than 9 weeks, less than 8 weeks, less than 7 weeks, less than 6 weeks, less than 5 weeks, less than 4 weeks, less than 3 weeks, less than 2 weeks, or less than 1 week.

The instant invention also encompasses ex vivo methods for treating, inhibiting, and/or preventing a disease or disorder in a subject. In certain embodiments, the method comprises administering at least two immune modulators to cells (e.g., immune cells, T cells, Tregs, precursor cells, pre-T cells, bone marrow cells, peripheral blood mononuclear cells (PBMCs)) and then administering the treated cells to the subject (e.g., as a cell-based therapy). In certain embodiments, the cells are autologous. In certain embodiments, the method further comprises obtaining the cells from the subject or a donor prior to treatment of the cells with the immune modulators. The immune modulators may be administered to the cells at the same time or they may be administered at different times (e.g., sequentially) as described hereinabove for the in vivo methods.

In accordance with the instant invention, compositions comprising at least two immune modulators are provided. In certain embodiments, the composition further comprises at least one carrier, particularly a pharmaceutically acceptable carrier. In certain embodiments, the composition further comprises at least one other neurodegenerative disease therapeutic.

Examples of immune modulators include, without limitation: GM-CSF (e.g., human recombinant GM-CSF, molgramostim, sargramostim), GM-CSF analogs, TGF-β, IL-10, IL-2, glatiramer acetate, anti-CD3 antibodies, anti-CD28 antibodies, bee venom phospholipase A₂ (bvPLA₂), rapamycin, histone deacetylase inhibitors, vasoactive intestinal peptide (VIP), VIP analogs and VIP receptor-2 (VIPR2, also known as VPAC2) agonists (e.g., LBT-3627). The immune modulators may be of the species of the subject being treated (e.g., human). In certain embodiments, at least one of the immune modulators is GM-CSF. In certain embodiments, at least one of the immune modulators is IL-2. In certain embodiments, the at least two immune modulators comprise GM-CSF and IL-2. In certain embodiments, the IL-2 is a low dose IL-2.

Immune modulators may comprise a protein, peptide, gene, small molecule, or oligonucleotide (e.g., mRNA or DNA). In certain embodiments, the immune modulator is a protein or polypeptide. In certain embodiments, the immune modulator is a nucleic acid molecule encoding the protein immune modulator. In certain embodiments, the nucleic acid molecule(s) encoding the protein immune modulator(s) is contained within a vector(s) (e.g., expression vector(s)) which may be administered to the subject. Examples of vectors for expressing the molecules of the invention include, without limitation, plasmids and viral vectors (e.g., adeno-associated viruses (AAVs), retroviruses, and lentiviruses). The expression vectors of the instant invention may employ a strong promoter, a constitutive promoter, and/or a regulated promoter. In certain embodiments, the promoter is cell-type specific. In certain embodiments, the nucleic acid molecules are expressed transiently.

In certain embodiments, the immune modulator(s) is formulated or packaged in a delivery vehicle. Examples of delivery vehicles include, without limitation: liposomes, dendrimers, microparticles, and/or nanoparticles. In certain embodiments, the formulation or delivery vehicle in a slow-release formulation or delivery vehicle.

The instant invention also encompasses kits comprising a first composition comprising a first immune modulator and a second composition comprising a second immune modulator. Generally, the term “kit” refers to a delivery system or enclosure (e.g., package or box) for delivering materials such as a combination of components, compositions, and/or packaging. For example, the kit may be a packaged product which packaging includes two or more independent containers or bottles, each containing a different composition. In certain embodiments, the kit includes one or more enclosures (e.g., boxes) containing the compositions and/or supporting materials (e.g., written instructions). In certain embodiments, each of the compositions further comprises at least one carrier, particularly a pharmaceutically acceptable carrier. In certain embodiments, one or more of the compositions further comprises at least one other neurodegenerative disease therapeutic. In certain embodiments, the kit further comprises a composition comprising at least one other neurodegenerative disease therapeutic and at least one carrier, particularly a pharmaceutically acceptable carrier.

Examples of immune modulators include, without limitation: GM-CSF (e.g., human recombinant GM-CSF, molgramostim, sargramostim), GM-CSF analogs, TGF-β, IL-10, IL-2, glatiramer acetate, anti-CD3 antibodies, anti-CD28 antibodies, rapamycin, histone deacetylase inhibitors, vasoactive intestinal peptide (VIP), VIP analogs and VIP receptor-2 (VIPR2, also known as VPAC2) agonists (e.g., LBT-3627). The immune modulators may be of the species of the subject being treated (e.g., human). In certain embodiments, at least one of the immune modulators is GM-CSF. In certain embodiments, at least one of the immune modulators is IL-2. In certain embodiments, the at least two immune modulators comprise GM-CSF and IL-2. In certain embodiments, the IL-2 is a low dose IL-2.

Immune modulators may comprise a protein, peptide, small molecule, or oligonucleotide (e.g., mRNA or DNA). In certain embodiments, the immune modulator is a protein or polypeptide. In certain embodiments, the immune modulator is a nucleic acid molecule encoding the protein immune modulator. In certain embodiments, the nucleic acid molecule(s) encoding the protein immune modulator(s) is contained within a vector(s) (e.g., expression vector(s)) which may be administered to the subject. Examples of vectors for expressing the molecules of the invention include, without limitation, plasmids and viral vectors (e.g., adeno-associated viruses (AAVs), retroviruses, and lentiviruses). The expression vectors of the instant invention may employ a strong promoter, a constitutive promoter, and/or a regulated promoter. In certain embodiments, the promoter is cell-type specific. In certain embodiments, the nucleic acid molecules are expressed transiently.

In certain embodiments, the immune modulator(s) is formulated or packaged in a delivery vehicle. Examples of delivery vehicles include, without limitation: liposomes, dendrimers, microparticles, and/or nanoparticles. In certain embodiments, the formulation or delivery vehicle in a slow-release formulation or delivery vehicle.

In accordance with an aspect of the instant invention, compositions and methods for increasing and/or inducing Treg function, activity, frequency/number, and/or development. The method may be in vitro or in vivo. In certain embodiments, the method comprises administering at least two immune modulators to the subject or cells (e.g., immune cells, T cells, Tregs, precursor cells, pre-T cells, bone marrow cells, peripheral blood mononuclear cells (PBMCs)). Examples of immune modulators include, without limitation: GM-CSF (e.g., human recombinant GM-CSF, molgramostim, sargramostim), GM-CSF analogs, TGF-β, IL-10, IL-2, glatiramer acetate, anti-CD3 antibodies, anti-CD28 antibodies, rapamycin, histone deacetylase inhibitors, vasoactive intestinal peptide (VIP), VIP analogs and VIP receptor-2 (VIPR2, also known as VPAC2) agonists (e.g., LBT-3627). The immune modulators may be of the species of the subject being treated (e.g., human). In certain embodiments, at least one of the immune modulators is GM-CSF. In certain embodiments, at least one of the immune modulators is IL-2. In certain embodiments, the at least two immune modulators comprise GM-CSF and IL-2. In certain embodiments, the IL-2 is a low dose IL-2. In certain embodiments, a low dose IL-2 is less than 5 million international units per day (MIU/day), less than 4 MIU/day, less than 3 MIU/day, less than 2 MIU/day, or less than 1 MIU/day. In certain embodiments, a low dose IL-2 is about 0.1 to about 5 MIU/day, about 0.5 to about 5 MIU/day, about 0.5 to about 3 MIU/day, or about 1 to about 3 MIU/day.

Immune modulators may comprise a protein, peptide, small molecule, or oligonucleotide (e.g. mRNA or DNA). In certain embodiments, the immune modulator is a protein or polypeptide. In certain embodiments, the immune modulator is a nucleic acid molecule encoding the protein immune modulator. In certain embodiments, the nucleic acid molecule(s) encoding the protein immune modulator(s) is contained within a vector(s) (e.g., expression vector(s)) which may be administered to the subject. Examples of vectors for expressing the molecules of the invention include, without limitation, plasmids and viral vectors (e.g., adeno-associated viruses (AAVs), retroviruses, and lentiviruses). The expression vectors of the instant invention may employ a strong promoter, a constitutive promoter, and/or a regulated promoter. In certain embodiments, the promoter is cell-type specific. In certain embodiments, the nucleic acid molecules are expressed transiently.

In certain embodiments, the immune modulator(s) is formulated or packaged in a delivery vehicle. Examples of delivery vehicles include, without limitation: liposomes, dendrimers, microparticles, and/or nanoparticles. In certain embodiments, the formulation or delivery vehicle in a slow-release formulation or delivery vehicle.

In certain embodiments, at least one of the immune modulators is GM-CSF. In certain embodiments, at least one of the immune modulators is IL-2. In certain embodiments, at least GM-CSF and IL-2 are administered. In certain embodiments, the immune modulators are administered to the subject or cell in a composition(s) further comprising at least one carrier, particularly at least one pharmaceutically acceptable carrier. In certain embodiments, the method further comprises administering at least one other neurodegenerative disease therapeutic.

The immune modulators may be administered at the same time or they may be administered at different times (e.g., sequentially). In certain embodiments, the at least two immune modulators are administered at the same time (e.g., in the same composition). In certain embodiments, the at least two immune modulators are administered sequentially. In certain embodiments, the at least two immune modulators are administered at least partially at the same time (e.g., their administration time frames overlap at least partially). In certain embodiments, the immune modulators are administered in the same composition when administered at the same time. In certain embodiments, the immune modulators are administered in separate compositions (e.g., one immune modulator per composition) when administered at the same time.

In certain embodiments, the method comprises administering a first immune modulator to the subject or cell for a first period of time (e.g., from the first to last administration of the first immune modulator) and a second immune modulator to the subject or cell for a second period of time (e.g., from the first to last administration of the second immune modulator). Generally, the first and second immune modulators are not the same immune modulator. In certain embodiments, the method further comprises administering a third or more immune modulator at a third or more period of time. In certain embodiments, the first period of time at least partially or completely overlaps with the second period of time. In certain embodiments, the first period of time does not overlap with the second period of time. In certain embodiments, the first period of time is before or at least begins before the second period of time. In certain embodiments, the first period of time is before or prior to the second period of time and does not overlap with the second period of time. In certain embodiments, the second immune modulator is IL-2 and is administered, at least partially or completely, after or later than the first immune modulator. In certain embodiments, the first immune modulator is GM-CSF and is administered, at least partially or completely, prior to or before the second immune modulator. In certain embodiments, the first immune modulator is GM-CSF, the second immune modulator is IL-2, and GM-CSF is administered, at least partially or completely, prior to or before the administration of IL-2.

When the first period of time does not overlap with the second period of time, the time in between the first period of time and the second period of time may be less than 2 weeks, less than one week, less than 6 days, less than 5 days, less than 4 days, less than 3 days, less than 2 days, or less than one day. In certain embodiments, the first period of time is less than 10 weeks, less than 9 weeks, less than 8 weeks, less than 7 weeks, less than 6 weeks, less than 5 weeks, less than 4 weeks, less than 3 weeks, less than 2 weeks, or less than 1 week. In certain embodiments, the second period of time is less than 10 weeks, less than 9 weeks, less than 8 weeks, less than 7 weeks, less than 6 weeks, less than 5 weeks, less than 4 weeks, less than 3 weeks, less than 2 weeks, or less than 1 week.

The compounds of the instant invention will generally be administered to a patient as a pharmaceutical preparation. The term “patient” as used herein refers to human or animal subjects. These compounds may be employed therapeutically, under the guidance of a physician for the treatment of cancer.

The pharmaceutical preparation comprising the compounds of the invention may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the compounds in the chosen medium may be varied and the medium may be chosen based on the desired route of administration of the pharmaceutical preparation. Except insofar as any conventional media or agent is incompatible with the compounds to be administered, its use in the pharmaceutical preparation is contemplated.

The dose and dosage regimen of the compounds according to the invention that is suitable for administration to a particular patient may be determined by a physician considering the patient's age, sex, weight, general medical condition, and the specific condition and severity thereof for which the compound is being administered. The physician may also consider the route of administration of the compound, the pharmaceutical carrier with which the compounds may be combined, and the compounds' biological activity.

Selection of a suitable pharmaceutical composition will also depend upon the mode of administration chosen. For example, the compositions of the invention may be administered by direct injection or intravenously. In this instance, a pharmaceutical composition comprises the components dispersed in a medium that is compatible with the site of injection.

Compositions of the instant invention may be administered by any method. For example, the compositions of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In certain embodiments, the methods of administration include but are not limited to: parenteral, subcutaneous, oral, topical, inhalation or intranasal, pulmonary, rectal, vaginal, intravenous, intraperitoneal, intrathecal, intracerebral, epidural, intramuscular, intradermal, or intracarotid administration. In a particular embodiment, the compositions are administered intramuscularly, subcutaneously, or to the bloodstream (e.g., intravenously). Pharmaceutical compositions for injection are known in the art. If injection is selected as a method for administering the composition, steps must be taken to ensure that sufficient amounts of the components reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing compounds of the present invention as the active ingredient in intimate admixture with a pharmaceutical carrier can be prepared according to conventional pharmaceutical compounding techniques. The carrier may take a wide variety of forms depending on the form of preparation desired for administration. In preparing the compound in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric-coated by standard techniques. For parenterals, the carrier will usually comprise sterile water, though other ingredients, for example, to aid solubility or for preservative purposes, may be included. Injectable suspensions may also be prepared, in which case appropriate liquid carriers, suspending agents and the like may be employed.

Compositions of the invention may be formulated in dosage unit form for ease of administration and uniformity of dosage. Dosage unit form, as used herein, refers to a physically discrete unit of the pharmaceutical composition appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. For example, the appropriate dosage unit for the administration of the composition may be determined by evaluating the toxicity of the composition in animal models. Various concentrations of the components in composition may be administered to mice or other mammals, and the minimal and maximal dosages may be determined based on the beneficial results and side effects observed as a result of the treatment.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of the compounds of the invention may be determined by evaluating the toxicity of the compounds in animal models. Various concentrations of the compounds of the instant invention may be administered to mice with transplanted human tumors, and the minimal and maximal dosages may be determined based on the results of significant reduction of tumor size and side effects as a result of the treatment. Appropriate dosage unit may also be determined by assessing the efficacy of the compounds in combination with other standard anti-cancer drugs. The dosage units of the compounds may be determined individually or in combination with each anti-cancer treatment according to greater shrinkage and/or reduced growth rate of tumors.

The compositions comprising the compounds of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### Definitions

The following definitions are provided to facilitate an understanding of the present invention.

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E.W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., neurodegenerative disease) resulting in a decrease in the probability that the subject will develop the condition.

A “therapeutically effective amount” of a compound or a pharmaceutical composition refers to an amount effective to prevent, inhibit, treat, or lessen the symptoms of a particular disorder or disease. The treatment of a neurodegenerative disease herein may refer to curing, relieving, and/or preventing the neurodegenerative disease, the symptom(s) of it, or the predisposition towards it.

As used herein, the term “subject” refers to an animal, particularly a mammal, particularly a human.

As used herein, a “biological sample” refers to a sample of biological material obtained from a subject, preferably a human subject, including a tissue, a tissue sample, a cell sample, a tumor sample, and a biological fluid (e.g., blood, urine, or amniotic fluid).

An “antibody” or “antibody molecule” is any immunoglobulin, including antibodies and fragments thereof, that binds to a specific antigen. Antibody fragments include, without limitation, antigen-binding immunoglobulin fragments including, without limitation: single domain (dAb; e.g., single variable light or heavy chain domain), Fab, Fab′, F(ab′)2, and F (v); and fusions (e.g., via a linker) of these immunoglobulin fragments including, without limitation: scFv, scFv2, scFv-Fc, minibody, diabody, triabody, and tetrabody.

With respect to antibodies, the term “immunologically specific” refers to antibodies that bind to one or more epitopes of a protein or compound of interest, but which do not substantially recognize and bind other molecules in a sample containing a mixed population of antigenic biological molecules.

The term “vector” refers to a carrier nucleic acid molecule (e.g., RNA or DNA) into which a nucleic acid sequence can be inserted, e.g., for introduction into a host cell where it may be expressed and/or replicated. An “expression vector” is a specialized vector that contains a gene or nucleic acid sequence with the necessary operably linked regulatory regions needed for expression in a host cell. The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector.

The following example provides illustrative methods of practicing the instant invention, and is not intended to limit the scope of the invention in any way.

### EXAMPLE

Parkinson's disease is the most common neurodegenerative motor disorder (Tysnes, et al. (2017) J. Neural. Transm., 124:901-5). The characteristic phenotype of the disorder includes tremors at rest and bradykinesia that result from loss of the neurotransmitter dopamine, which is due, in large part, to the loss of dopamine-synthesizing neurons that originate in the substantia nigra (SN) pars compacta and 10 innervate to the striatum. As one of many synucleinopathies, intraneuronal inclusions of misfolded and aggregated α-synuclein (α-syn) and ubiquitin accumulate to form Lewy bodies, which are hallmarks of PD and considered posthumously diagnostic for PD (Li, et al. (2008) Nat. Med., 14:501-3; Mendez, et al. (2008) Nat. Med., 14:507-9). A second characteristic hallmark of PD is chronic inflammation mediated by innate immune cells, such as microglia and infiltrating macrophages. Abundant evidence indicates that high levels of inflammation lead to an increased oxidative state in PD and play a major role in disease progression and possibly etiology (Benner, et al. (2008) PLOS One 3: e1376; Gelders, et al. (2018) J. Immunol. Res., 2018:4784268; Mosley, et al. (2012) Cold Spring Harb. Perspect. Med., 2: a009381). Activated microglia secrete neurotoxic mediators and sufficiently increase oxidative stress to induce misfolding and modification of α-syn, which is secreted or released into the extraneuronal environment upon cell injury or death, and activates surrounding microglia to perpetuate a chronic inflammatory state. Moreover, inflammatory molecules secreted by microglia also upregulate expression of monomeric α-syn in neurons and promote its aggregation (Dutta, et al. (2021) Nat. Commun., 12:5382). Activated microglia can also elevate astrocytes to a reactive A1 neurotoxic phenotype and play an important role in persistent chronic neuroinflammation and α-syn aggregate formation (Liddelow, et al. (2017) Immunity 46:957-67; Miyazaki, et al. (2020) Cells 9:2623; Yun, et al. (2018) Nat. Med., 24:931-8; Bantle, et al. (2021) Exp. Neurol., 346:113845; Liddelow, et al. (2017) Nature 541:481-7). Notably, neuronal exosomes that contain monomeric and oligomeric α-syn are associated with spreading pathogenic α-syn species to other neurons and glia that, in turn, support persistent inflammatory states (Pascual, et al. (2020) Neural Regen. Res., 15:796-801; Volpicelli-Daley, et al. (2014) Nat. Protoc., 9:2135-46; Alvarez-Erviti, et al. (2011) Neurobiol. Dis., 42:360-7; Jiang, et al. (2020) J. Neurol. Neurosurg. Psychiatry 91:720-9; Fussi, et al. (2018) Cell Death Dis., 9:757; Dutta, et al. (2021) Acta Neuropathol., 142:495-511). In contrast, immune forces that control and regulate chronic inflammation are diminished in PD (Mosley, et al. (2012) Cold Spring Harb. Perspect. Med., 2: a009381; Alvarez-Luquin, et al. (2019) J. Neuroinflammation 16:212; Reynolds, et al. (2010) J. Immunol., 184:2261-71; Saunders, et al. (2012) J. Neuroimmune Pharmacol., 7:927-38; Schwab, et al. (2020) Neurobiol. Dis., 137:104760). Indeed, regulatory T cells (Tregs), which maintain active immunological tolerance and attenuate inflammatory responses, are significantly diminished in PD patients, while activated pro-inflammatory myeloid and effector T cells (Teffs) are increased (Saunders, et al. (2012) J. Neuroimmune Pharmacol., 7:927-38; Gendelman et al. (2017) NPJ Parkinsons Dis., 3:10; Olson, et al. (2021) EBioMedicine 67:103380; Thome, et al. (2021) NPJ Parkinson's Dis 7:41).

Tregs can transform pro-inflammatory, neurotoxic environments to more anti-inflammatory and neurotrophic environments, wherein dopaminergic neurons are rescued or spared from a degenerative fate (Reynolds, et al. (2010) J. Immunol., 184:2261-71; Gendelman et al. (2017) NPJ Parkinsons Dis., 3:10; Olson, et al. (2021) EBioMedicine 67:103380; Mosley, et al. (2019) Front. Cell Neurosci., 13:421; Olson, et al. (2021) Biomaterials 272:120786; Olson, et al. (2020) Neurotherapeutics 17:1861-77; Potter, et al. (2021) Alzheimers Dement., 7: e12158). Indeed, immunomodulatory agents such as granulocyte-macrophage colony stimulating factor (GM-CSF) increase Treg numbers and activity with parallel neuroprotective activities in PD and Alzheimer's disease (AD) (Reynolds, et al. (2010) J. Immunol., 184:2261-71; Gendelman et al. (2017) NPJ Parkinsons Dis., 3:10; Olson, et al. (2021) EBioMedicine 67:103380; Thome, et al. (2021) NPJ Parkinson's Dis 7:41; Olson, et al. (2021) Biomaterials 272:120786; Olson, et al. (2020) Neurotherapeutics 17:1861-77; Potter, et al. (2021) Alzheimers Dement., 7: e12158; Kiyota, et al. (2018) J. Neuroimmunol., 319:80-92; Kosloski, et al. (2013) J. Neuroimmunol., 265:1-10). Many regulatory mechanisms of induced Tregs (iTregs) utilize known pathways for improving disease outcomes; however, whether all pathways are operative in PD have yet to be determined. Therefore, to compensate Treg deficits, optimal levels of iTregs or iTreg activity could better control disease-initiating inflammatory activities and mitigate disease progression. Thus, novel therapeutic approaches would utilize immunomodulatory agents to induce Tregs to restore diminished numbers and/or activity, attenuate neuroinflammation, and re-establish or maintain immune tolerance with intervention of disease progression in PD patients. Indeed, while GM-CSF seems to satisfy those directives, the GM-CSF signaling pathway that induces Tregs seems not to be one of direct Treg interaction (Olson, et al. (2021) Biomaterials 272:120786; Schutt, et al. (2018) Mol. Neurodegener., 13:26).

Interleukin-2 (IL-2) has essential defined roles and direct interaction in survival and expansion of new or existing Tregs. IL-2 upregulates forkhead box P3 (FoxP3) expression during Treg development in the thymus and is involved in Treg differentiation, lineage stability, proliferation, and function (Zorn, et al. (2006) Blood 108:1571-9). Tregs constitutively express the high-affinity receptor for IL-2, while its expression by other subsets of T cells is induced after activation (Chinen, et al. (2016) Nat. Immunol., 17:1322-33). IL-2 signaling is also a key component by which CD4+CD25+FoxP3+iTregs exhibit functional dominance and can outgrow other T-cell types that typically express lower levels of the IL-2 receptor a chain (CD25) (Hotta-Iwamura, et al. (2018) J. Autoimmun., 90:39-48; Dong, et al. (2021) JCI Insight 6: e147474; MacMillan, et al. (2021) (2021) Blood Adv., 5:1425-36; Hui, et al. (2021) Front. Immunol., 12:619932). Therefore, in the steady state, functional iTregs respond better to IL-2 than other T cells and do so in a direct manner, rather than via intermediary cell interactions (Abba, et al. (2018) Sci. Immunol., 3: eaat1482). This may account for the ability of low doses of IL-2 to preferentially increase Treg numbers without global immune activation of other T cell types (Grinberg-Bleyer, et al. (2010) J. Exp. Med., 207:1871-8; Tang, et al. (2008) Immunity 28:687-97; Wang, et al. (2021) Int. Immunopharmacol., 97:107663). However, whether IL-2 selectively induces new Tregs or merely expands existing, but possibly deficient, Tregs is currently unknown and untested in PD. Therapies such as low-dose IL-2 or IL-2/anti-IL-2 antibody complexes may preferentially expand Treg populations as a treatment for chronic inflammatory autoimmune diseases (Abbas, A. K. (2020) Am. J. Pathol., 190:1776-81). Some neuroprotective effects of low-dose IL-2 treatment have been found in AD mice (Alves et al. (2017) Brain 140:826-42) and in amyotrophic lateral sclerosis (ALS) patients (Giovannelli, et al. (2021) Brain Commun., 3: fcab141). Nevertheless, no study thus far has investigated the effects of low-dose IL-2 in PD or pre-clinical PD models or the synergistic effects of low-dose IL-2 with other immune modulators such as GM-CSF.

Herein, the effect of the combination therapy of IL-2, particularly low-dose IL-2, and GM-CSF on Treg expansion was studied.

### Materials and Methods

C57BL/6 male mice (5-6 weeks old) were purchased from Jackson Laboratories (Bar Harbor, ME). Mice were weighed and divided into four treatment groups (n=3 per group): 1) PBS (phosphate-buffered saline; control), 2) GM-CSF (0.1 mg/kg), 3) low dose IL-2 (2.5×10⁴ IU; human recombinant interleukin-2 (PeproTech, Cranbury, NJ)), and 4) GM-CSF (0.1 mg/kg) and low dose IL-2 (2.5×10⁴ IU). Following a period of acclimation, mice were injected intraperitoneally (i.p.) with daily GM-CSF (Group 4) (Days 0-4). On Days 5-9, daily treatment began for Groups 1-3 and IL-2 treatment began for Group 4. On Day 10, the mice were sacrificed, blood was collected, and spleen and lymph nodes were harvested.

Following treatment, whole blood, lymph nodes and spleens were collected to determine T-cell profiles via flow cytometric analysis. Peripheral blood, lymph nodes, and splenocytes were fluorescently labeled using antibodies against cell surface antigens including CD3, CD4, CD25, CD8, CD19, CD127, ICOS, GITR, CD39, CD62L, CD44, GITR, and CTLA-4, and for the intracellular markers FoxP3 and STAT5. Whole blood, lymph nodes, and splenocytes were labeled with PerCP Cyanine 5.5-anti-CD3 (eBioscience/Thermo Fisher Scientific, Waltham, MA), APC-anti-CD19 (eBiosciences), PE-anti-CD25 (eBiosciences), PE-cyanine7-anti-CD4 (eBiosciences), PerCP-eFluor710-anti-CD39 (eBiosciences), Alexa Fluor™ 488-anti-CD278 (ICOS) (Biolegend, San Diego, CA), FITC-anti-CD127 (Biolegend), FITC-anti-CD44, Alexa Fluor™ 488-anti-CTLA-4, Alexa Flour™ 700-anti-GITR, FITC-anti-CD8, and PerCP Cyanine 5.5-anti-CD62L. Intracellular staining was performed following permeabilization for 45 minutes at 4° C. Cells were labeled with APC-anti-FoxP3 (eBioscience) or anti-STAT5-Pacific Blue. Samples were analyzed with an LSRII flow cytometer and FACSDiva™ Software (BD Biosciences, San Diego, CA).

All values are expressed as mean±SEM using ANOVA (mean±SEM, n=3). *=p≤0.05; **=p≤0.01; *** p≤0.001. Data were analyzed using GraphPad Prism 9.3.1 software (La Jolla, CA).

### Results

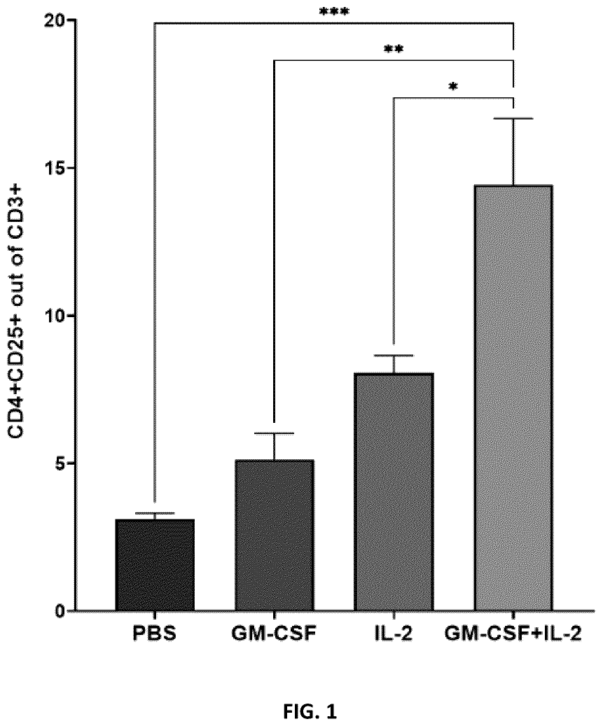

CD4+CD25+ Tregs in blood samples were quantified by flow cytometry. As seen in FIG. 1, the combination therapy of GM-CSF and IL-2 induced a dramatic increase in the number of Tregs. The synergistic increase in Tregs was greater than an additive effect of GM-CSF and IL-2 administered individually.

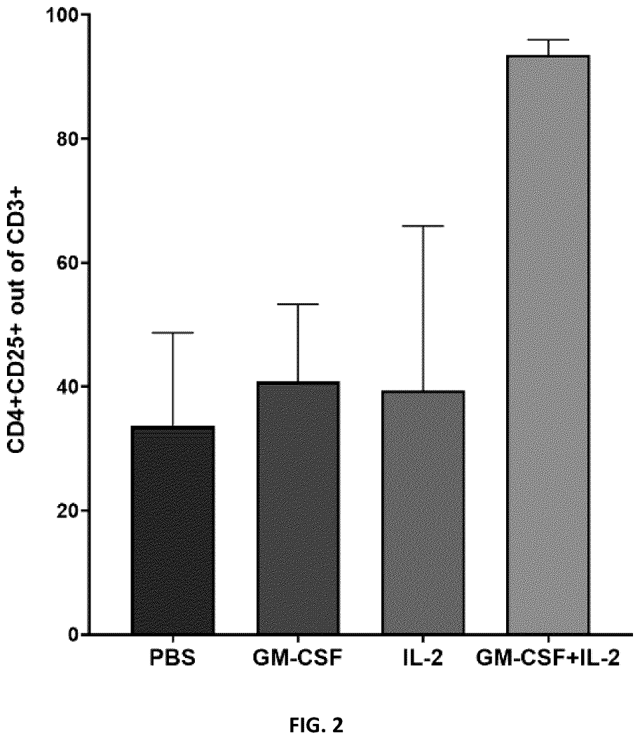

Similarly, CD4+CD25+ Tregs were dramatically increased in the harvested lymph nodes from mice treated with both GM-CSF and IL-2. As seen in FIG. 2, GM-CSF and IL-2 yielded only a mild increase in CD4+CD25+ Tregs when administered alone. However, a synergistic increase in CD4+CD25+ Tregs was observed with the combination therapy.

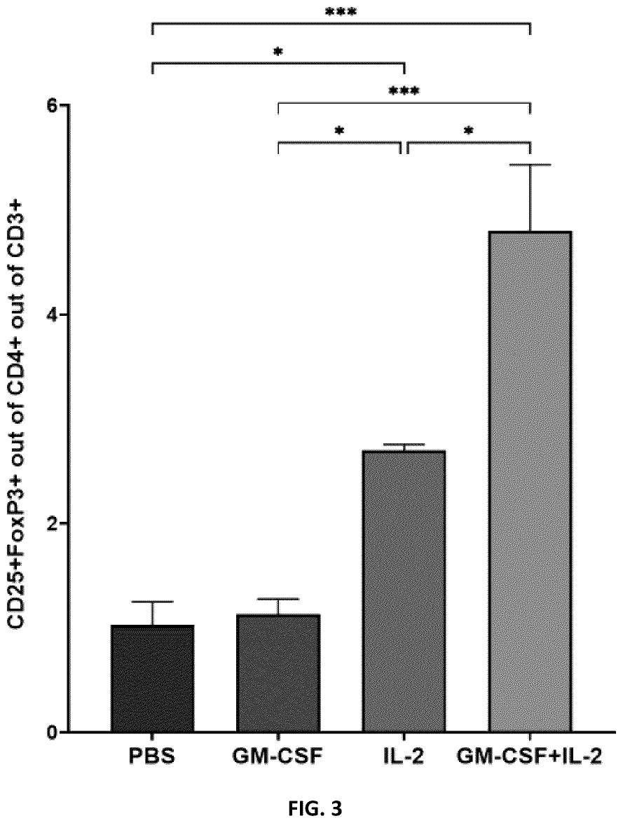

FoxP3 is a marker for murine Tregs. Harvested spleen were examined for CD4+CD25+FOXP3+ Tregs. As seen in FIG. 3, GM-CSF alone did not induce CD4+CD25+FOXP3+ Tregs. While IL-2 alone led to an increase in CD4+CD25+FOXP3+ Tregs, the combination therapy of GC-CSF and IL-2 led to a dramatic increase in CD4+CD25+FOXP3+ Tregs.

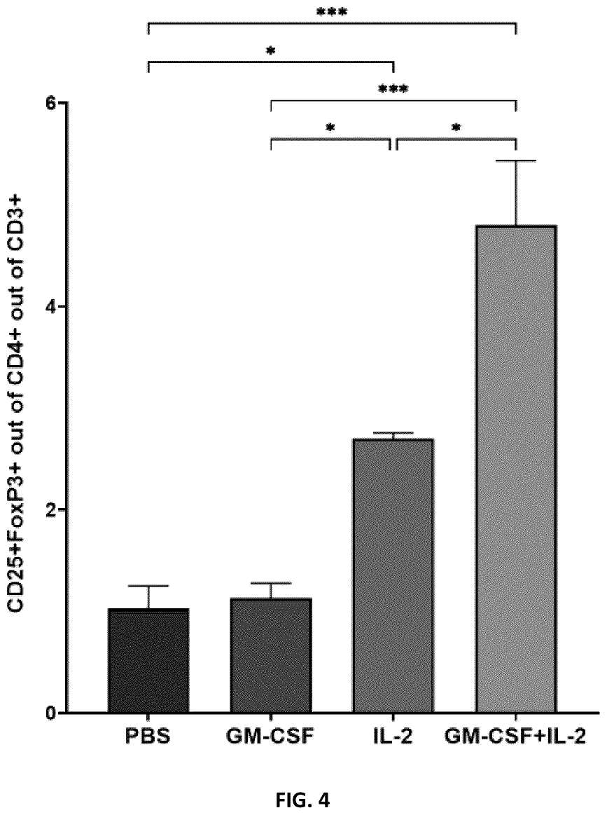

Similar results were observed in blood samples. As seen in FIG. 4, GM-CSF alone did not induce CD4+CD25+FOXP3+ Tregs. While IL-2 alone led to an increase in CD4+CD25+FOXP3+ Tregs, the combination therapy of GC-CSF and IL-2 led to a dramatic increase in CD4+CD25+FOXP3+ Tregs.

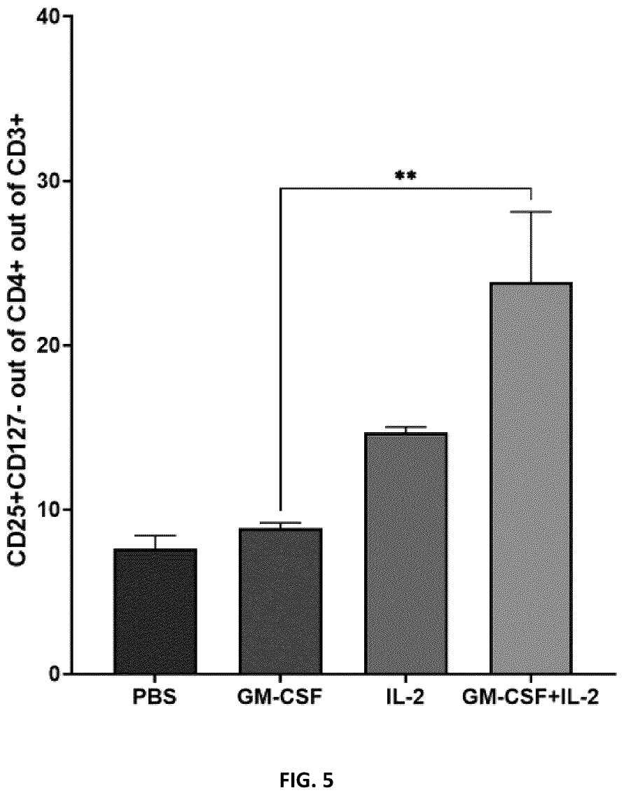

CD25+CD127− Tregs were also studied in blood and spleen. As seen in FIGS. 5 (blood) and 6 (spleen), GM-CSF alone did not induce CD25+CD127-Tregs. While IL-2 alone led to an increase in CD25+CD127− Tregs, the combination therapy of GC-CSF and IL-2 led to a dramatic increase in CD25+CD127− Tregs.

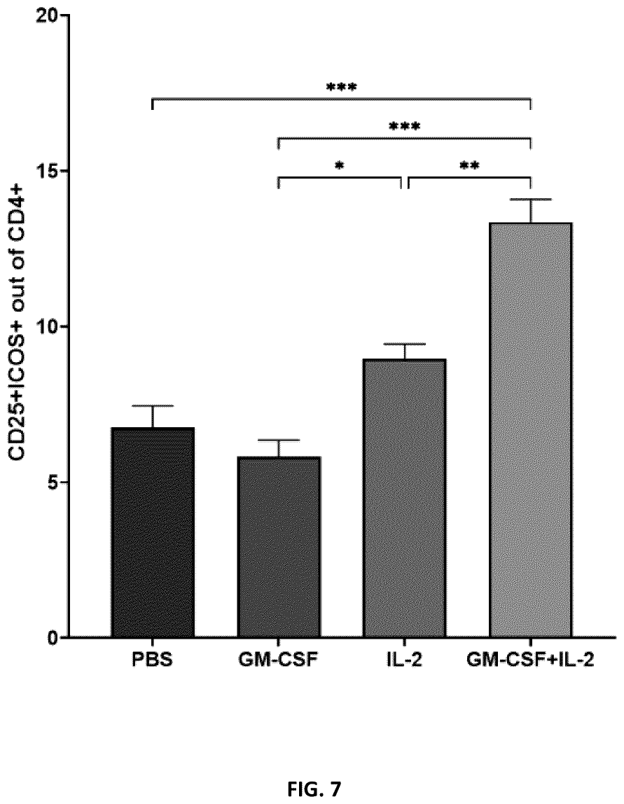

Inducible costimulator (ICOS) is a costimulatory molecule in the CD28 family. As seen in FIGS. 7 (blood) and 8 (spleen), GM-CSF alone and IL-2 alone did not significantly induce CD25+ICOS+ Tregs. In contrast, the combination therapy of GC-CSF and IL-2 led to a dramatic increase in CD25+ICOS+ Tregs.

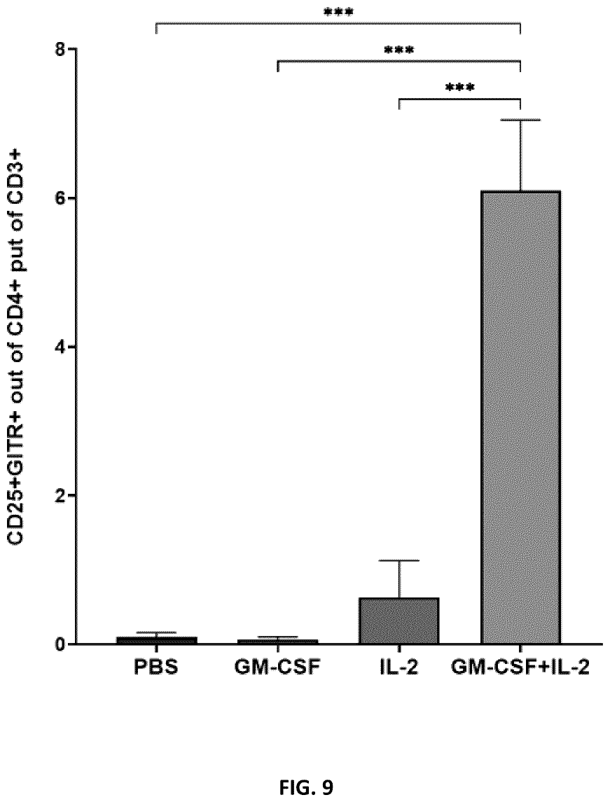

Treg cells express high levels of the glucocorticoid-induced tumor necrosis factor-related receptor (GITR). As seen in FIG. 9, GM-CSF alone did not induce CD25+GITR+ Tregs. While IL-2 alone led to a small increase in CD25+GITR+ Tregs, the combination therapy of GC-CSF and IL-2 led to a dramatic increase in CD25+GITR+ Tregs.

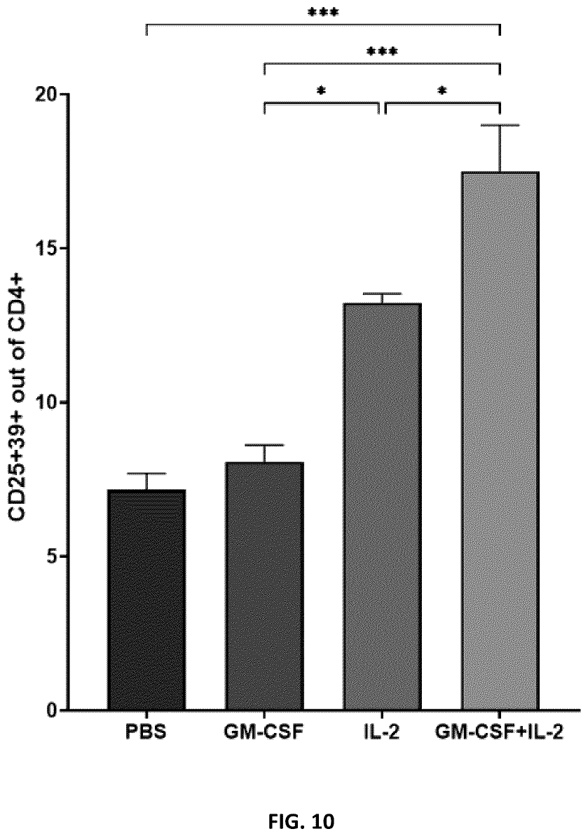

CD39 is expressed primarily by immune-suppressive FOXP3+ Tregs. As seen in FIG. 10, GM-CSF alone did not induce CD25+CD39+ Tregs. While IL-2 alone led to an increase in CD25+CD39+ Tregs, the combination therapy of GC-CSF and IL-2 led to a significant increase in CD25+CD39+ Tregs.

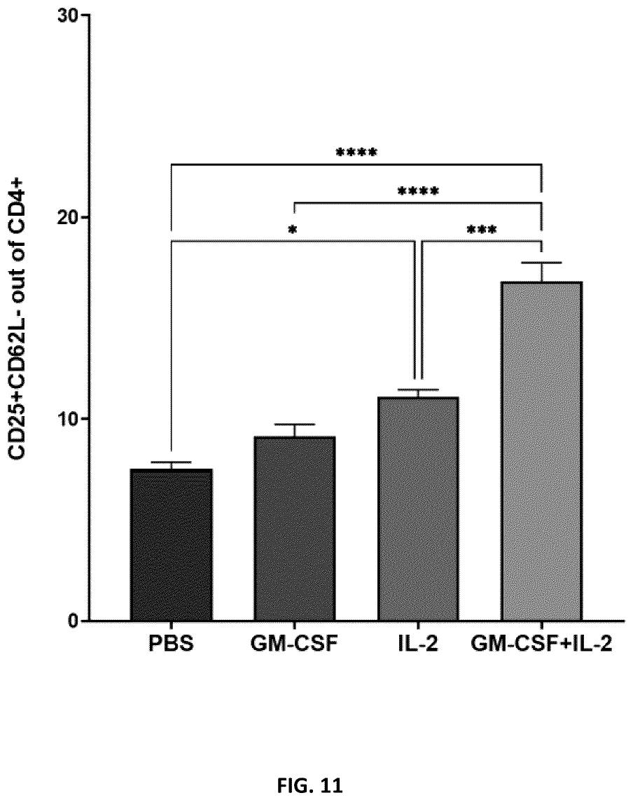

Harvested spleen were also analyzed for expression of CD62L. As seen in FIG. 11, GM-CSF alone did not significantly induce CD25+CD62L− Tregs. While IL-2 alone led to a small increase in CD25+CD62L− Tregs, the combination therapy of GC-CSF and IL-2 led to a dramatic increase in CD25+CD62L− Tregs.

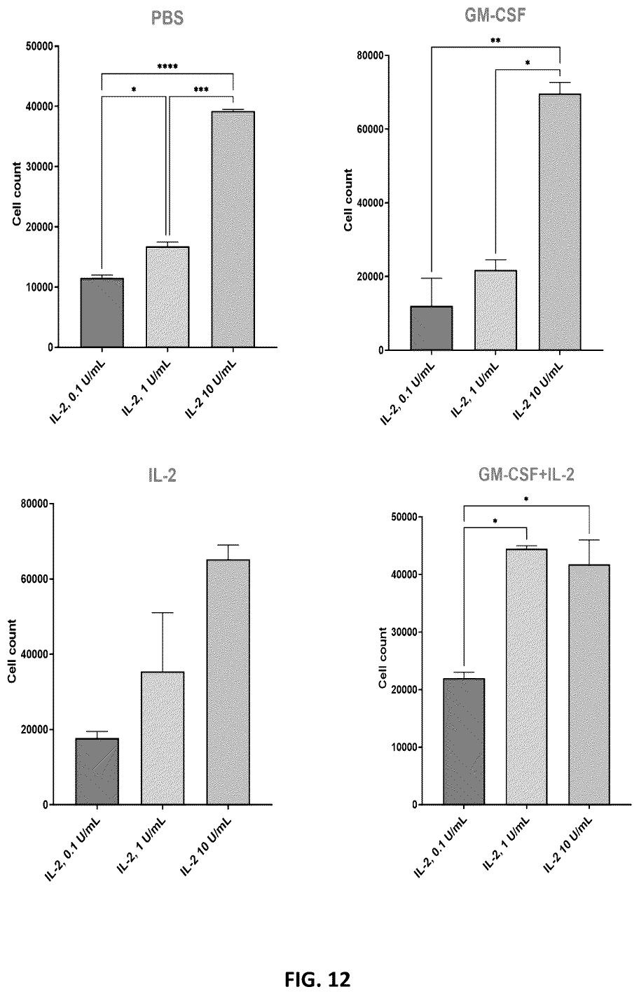

The in vitro induction of isolated Tregs with low doses of IL-2 was also studied. CD4+CD25+ cells were isolated from pooled lymph nodes and spleens of mice from the four treatment groups (PBS, GM-CSF, IL-2, IL-2+GM-CSF). The cells were seeded at 50,000 cells per well and cultured with low doses of IL-2:0.1 U/ml, 1 U/ml, or 10 U/ml. Cells were harvested after 7 days and flow cytometry analysis was performed. As seen in FIG. 12, CD4+CD25+ cells from mice treated with GM-CSF and IL-2 were more readily stimulated by low levels of IL-2 than the other treatment groups.

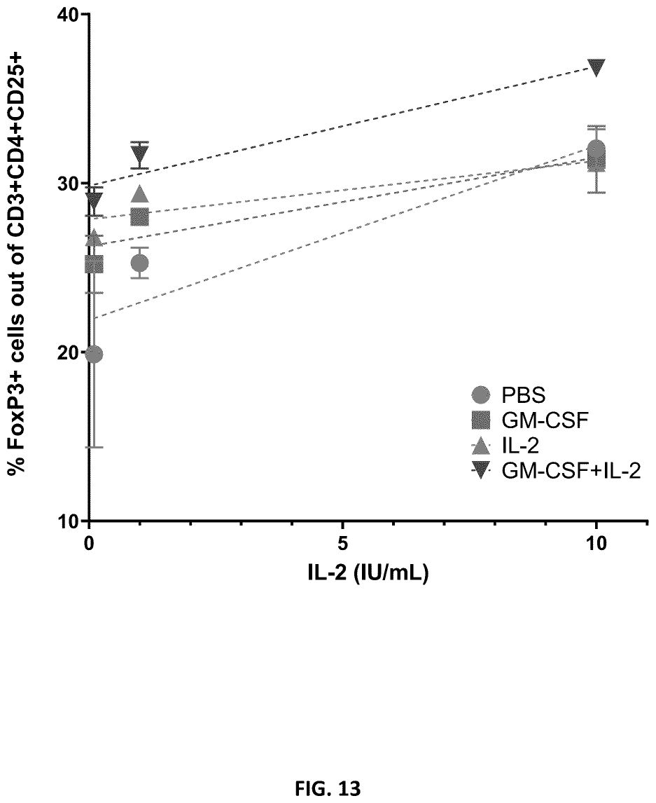

Lastly, the percentage of FoxP3+ cells of the in vitro IL-2-induced CD4+CD25+ cells was also determined. As seen in FIG. 13, cells from mice treated with IL-2 and GM-CSF had a greater percentage of FoxP3+ cells at each of the three levels of IL-2.

Herein, an in vivo study was performed to elucidate the impact of concomitant GM-CSF and IL-2, particularly low-dose IL-2, mediated transformation and expansion on Treg phenotypes in peripheral blood, spleen, and lymphoid tissues. Mouse spleen, lymph nodes and blood were profiled, which demonstrated synergistic effects with the treatment with GM-CSF and IL-2 compared to control (PBS) or treatment with IL-2 or GM-CSF alone. Treg cell markers were shown to be significantly expressed by flow cytometric analyses.

Experimental evidence in healthy mice demonstrated a 4 to 6-fold higher expression of percentages of Foxp3⁺CD4⁺CD25⁺ T-cells (regulatory T cells) in mice treated with both IL-2 and GM-CSF, compared to treatments with either cytokine alone or PBS. Other markers shown to define a suppressive Treg phenotype were increased as well. Percentages of CD25⁺CD127^{low} cells were significantly (up to 4 times) elevated, percentages of CD25⁺CD4⁺ regulatory T-cells that express CD39⁺ or ICOS⁺ markers were also doubled in the spleens of mice treated with both IL-2 and GM-CSF compared to PBS-treated mice.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of treating, inhibiting, and/or preventing a disease or disorder in a subject in need thereof, said method comprising administering at least two immune modulators to said subject.

2. The method of claim 1, wherein the disease or disorder is selected from the group consisting of a neurodegenerative disease, an autoimmune disease, an inflammatory disease, and cancer.

3. The method of claim 2, wherein the disease or disorder is Parkinson's disease.

4. The method of claim 1, wherein said immune modulators are selected from the group consisting of GM-CSF, GM-CSF analogs, TGF-β, IL-10, IL-2, glatiramer acetate, anti-CD3, anti-CD28, bee venom phospholipase A₂ (bvPLA₂), rapamycin, histone deacetylase inhibitors, vasoactive intestinal peptide (VIP), VIP analogs, and VIP receptor-2 agonists.

5. The method of any one of claims 1-4, wherein said immune modulators are administered simultaneously.

6. The method of any one of claims 1-4, wherein said immune modulators are administered sequentially.

7. The method of any one of claims 1-6, wherein at least one of said immune modulators is granulocyte-macrophage colony-stimulating factor (GM-CSF).

8. The method of any one of claims 1-6, wherein at least one of said immune modulators is interleukin-2 (IL-2).

9. The method of claim 8, wherein said IL-2 is low dose IL-2.

10. The method of any one of claims 1-9, wherein said at least two immune modulators comprise GM-CSF and IL-2.

11. The method of claim 1, wherein said method comprises administering granulocyte-macrophage colony-stimulating factor (GM-CSF) to said subject and then administering interleukin-2 (IL-2) to said subject.

12. The method of claim 11, wherein the disease or disorder is Parkinson's disease.

13. The method of claim 12, wherein said IL-2 is low dose IL-2.

14. A composition comprising at least two immune modulators and at least one pharmaceutically acceptable carrier.

15. The composition of claim 14, wherein said immune modulators are selected from the group consisting of GM-CSF, GM-CSF analogs, TGF-β, IL-10, IL-2, glatiramer acetate, anti-CD3, anti-CD28, bee venom phospholipase A₂ (bvPLA₂), rapamycin, histone deacetylase inhibitors, vasoactive intestinal peptide (VIP), VIP analogs and VIP receptor-2 agonists.

16. The composition of claim 14, wherein said composition comprises granulocyte-macrophage colony-stimulating factor (GM-CSF) and interleukin-2 (IL-2).

17. A method for increasing and/or inducing regulatory T cells (Tregs) said method comprising administering at least two immune modulators to immune cells.

18. The method of claim 17, wherein said immune cells comprise T cells, pre-T cells, bone marrow cells, and/or peripheral blood mononuclear cells (PBMCs).

19. The method of claim 17, wherein said immune modulators are selected from the group consisting of GM-CSF, GM-CSF analogs, TGF-β, IL-10, IL-2, glatiramer acetate, anti-CD3, anti-CD28, rapamycin, histone deacetylase inhibitors, vasoactive intestinal peptide (VIP), VIP analogs, and VIP receptor-2 agonists.

20. The method of any one of claims 17-19, wherein said at least two immune modulators comprise GM-CSF and IL-2.

21. The method of claim 17, wherein said method comprises administering granulocyte-macrophage colony-stimulating factor (GM-CSF) to said immune cell and then administering interleukin-2 (IL-2) to said immune cell.
